(19) United States
(12) Patent Application Publication
(10) Pub. No.: US 2017/0027852 A1
(43) Pub. Date: Feb. 2, 2017

(54) Topical Skin Care Composition For Day Use

(71) Applicants: Sara Sweeney, Austin, TX (US); Fred H. Khoury, Chatsworth, CA (US)

(72) Inventors: Sara Sweeney, Austin, TX (US); Fred H. Khoury, Chatsworth, CA (US)

(73) Assignee: Truth Aesthetics LLC, Austin, TX (US)

(21) Appl. No.: 14/814,243

(22) Filed: Jul. 30, 2015

(51) Int. Cl. A61K 8/97; A61Q 19/08; A61K 8/64; A61K 8/73; A61K 8/44; A61K 8/67

(52) U.S. Cl. CPC A61K 8/97; A61K 8/445; A61K 8/678; A61K 8/64; A61K 8/676; A61K 8/73; A61Q 19/08

(57) ABSTRACT

Topical skin care composition for day use containing a combination of stabilized antioxidants, oat derived avenanthramide and biofunctional peptides and other optional ingredients for topical application for the treatment and prevention of skin damage due to environmental factors.

### BACKGROUND

This invention is related to a topical skin care composition as well as methods of using same. More specifically, the invention relates to a topical skin care composition for use during the day to protect against environmental hazards, pollutants and thermal and infra-red radiation.

The skin ages as a natural consequence of exposure to various environmental factors. Among these factors is exposure to air pollutants, as well as thermal and infra-red radiation. It is reported that over 85% of the visible signs of aging are due to the negative effect of these factors on the skin. Other factors that may play a part in the aging process of the skin include, for example, weathering of the skin, exposure to cigarette smoke and Ultra-violet radiation.

Most conventional cosmetic products merely temporarily mask the signs of aging, and do little to adequately protect the skin's collagen and elastin network from the effects of the environmental factors listed above. What has been needed, and heretofore unavailable, is a topically applied skin care formulation for use during the day that protects the skin from environmental factors that a sunscreen does not address while helping to improve skin resiliency and radiance, and which also addresses fine lines and wrinkles by protecting the skin's collagen and elastin network and preventing loss of protective bio-agents such as CoQ10. The present invention satisfies these and other needs.

### SUMMARY OF THE INVENTION

In its most general aspect, the invention includes a topical skin care composition comprising a combination of an aloe leaf extract, a stable, oil soluble form of Vitamin C, a palm tocotrienol/tocopherol complex, a bioengineered peptide to boost the natural endogenous synthesis of CoQ10, a glutathione-biomimetic antioxidant peptide, a peptide inhibitor of elastase and MMP-1, an exopolysaccharide, a knotgrass extract, a phyto-complex, an oat avenanthramide extract, an oat β-glucan, a soy based complex, an extract from dried apples, and a cosmetically acceptable carrier.

In another aspect, the various components described above are blended with a cosmetically acceptable carrier which may include purified water, oils, alcohols, glycols, and combinations thereof.

In yet another aspect, the topical skin care composition may further comprise additional ingredients such as penetration enhancers, humectants, lubricants, pharmaceutically active agents, color, fragrance, preservatives, antioxidants, chelators, neutralizers, amino acids, anti-inflammatory agents, anti-irritants, anti-tack agents, astringents, binders, catalysts, stabilizers, emollients, emulsifiers, surfactants, cell-signaling agents, essential oils, plant/botanical extracts, conditioners, film formers, gelling agents, foaming agents, exfoliants, vitamins, minerals, pH adjusters, proteins, peptides, tactile enhancers, saccharides, solvents or any combination thereof.

In still another aspect, the topical skin care composition may be formulated as a cream, lotion, serum, facial cleanser, toner, eye cream, sunscreen, stick, spray, impregnated personal care device, impregnated towelette, gel, fluid/liquid, soap, oil, butter, peel, scrub, mask, concentrate, or any other form known in the art.

In another aspect, the present invention includes a topical skin care composition comprising: (a) an oat derived avenanthramide extract; (b) an aloe barbadensis leaf extract; (c) a source of vitamin E; (d) a source of biofunctional peptides; (e) an ascorbic acid source; (f) a blend of anti-inflammation and anti-oxidant components; and (g) a cosmetically acceptable carrier.

In a further aspect, the invention includes a method of treating skin comprising applying a topical skin care composition comprising a combination of an aloe leaf extract, a stable, oil soluble form of Vitamin C, a palm tocotrienol/tocopherol complex, a bioengineered peptide to boost the natural endogenous synthesis of CoQ10, a glutathione-biomimetic antioxidant peptide, a peptide inhibitor of elastase and MMP-1, an exopolysaccharide, a knotgrass extract, a phyto-complex, an oat avenanthramide extract, an oat β-glucan, a soy based complex, an extract from dried apples, and a cosmetically acceptable carrier.

In still another aspect, the topical skin care composition is applied to the skin, often the face, which may, for example, but not limited to, have wrinkles, fine lines, uneven tone, loss of firmness, surface roughness, dark circles, under-eye puffiness, sun damage, redness, dryness, irritation, enlarged ports and combinations of all or some of the above. Alternatively, the topical skin care composition may be applied to the skin to prevent the occurrence of the various problems described above.

In yet another aspect, the topical skin care composition is applied to skin in an amount and for a period of time sufficient to treat the skin for the condition treated. In one alternative aspect, the topical skin care composition is applied at least once a day. In another alternative aspect, the topical skin care composition is applied more than once a day.

In still another aspect, a user of the topical skin care composition of the present invention cleanses his/her skin and gently pats the skin dry. A thin, even layer of the topical skin care composition of the present invention is applied to the face, neck, or other portions of the body. The topical skin care composition is then gently massaged into the skin. This process may be performed every morning, for example.

In yet another aspect, the invention includes a topical skin care composition comprising: an oat derived avenanthramide; an aloe barbadensis leaf extract; a source of vitamin E; a source of biofunctional peptides; an ascorbic acid source; a blend of anti-inflammation and anti-oxidant components; and a cosmetically acceptable carrier.

In one aspect, the source of vitamin E is a palm tocotrienol/tocopherol complex. In another aspect, the aloe barbadensis extract is present in the amount of 0.09% by weight to 0.11% by weight. In still another aspect, the source of ascorbic acid is tetrahexydecyl ascorbate. In an alternative aspect, the source of ascorbic acid is present in the amount of 0.05% by weight to 1.0% by weight. In yet another aspect, the biofunctional peptides are present in the amount of 0.05% by weight to 1.0% by weight.

In still another aspect, the oat derived avenanthramide extract is present in the amount of 0.1% by weight to 3.0% by weight. In an alternative aspect, the oat derived avenanthramide extract is present in the amount of 0.45% by weight to 0.55% by weight.

In yet another aspect, the topical skin care composition of the invention further comprises a soy based active complex. In another aspect, the topical skin care composition of the invention further comprises a phyto-complex component.

In another aspect, the topical skin care composition of the invention further comprises a source of flavonoids. In one alternative aspect, the source of flavonoids includes knotgrass extract.

In still another aspect, the blend of anti-inflammation and anti-oxidant components includes colloidal oatmeal. In another aspect, the blend of anti-inflammation and anti-oxidant components includes tocopherol. In yet another aspect, the blend of anti-inflammation and anti-oxidant components includes tocopherol and tocotrienol.

In yet another aspect, the source of biofunctional peptides includes pentapeptide-34 trifluoroacetate. In an alternative aspect, the source of biofunctional peptides includes dipeptide-4.

In a further aspect, the topical skin care composition further includes an exopolysaccharide. In another further aspect, the topical skin care composition includes an apple extract.

In another aspect, the present invention includes a method of treating the skin, comprising applying to the outer surface of the skin a composition comprising: an oat derived avenanthramide; an aloe barbadensis leaf extract; a source of vitamin E; a source of biofunctional peptides; an ascorbic acid source; a blend of anti-inflammation and anti-oxidant components; and a cosmetically acceptable carrier.

Other features and advantages of the present invention will become apparent from the following detailed description, taken in conjunction with the accompanying drawings, which illustrate, by way of example, the principles of the invention.

### DETAILED DESCRIPTION OF THE PREFERRED EMBODIMENTS

As used herein, the phrases “selected from the group consisting of,” “chosen from,” and the like include mixtures of the specified materials. Tunis such as “contains” and the like are meant to include “including at least” unless otherwise specifically noted.

Where a numerical limit or range is stated, the endpoints are included. Also, all values and subranges within a numerical limit or range are specifically included as if explicitly written out.

The various embodiments and compositions described herein are typically used by persons desiring to protect their skin from harmful environmental factors, or to repair skin that has been previously damaged by such factors. For example, persons using those compositions may seek to prevent damage to the skin caused by lack of hydration, inflammation or infrared radiation. Benefits of using the compositions of the present invention include retaining and/or restoring and/or improving physical and mechanical properties of the skin which includes smoothness, taughtness, resiliency and radiance.

The present invention is directed to a topical skin care composition containing a combination of an aloe leaf extract, a stable, oil soluble form of Vitamin C, a palm tocotrienol/tocopherol complex, a bioengineered biofunctional peptide to boost the natural endogenous synthesis of CoQ10, a glutathione-biomimetic antioxidant peptide, a peptide inhibitor of elastase and MMP-1, an exopolysaccharide, a knotgrass extract, a phyto-complex, an oat avenanthramide extract, an oat β-glucan, a soy based complex, and an extract from dried apples. The combination promotes rejuvenation of the skin and inhabits damage to skin caused by dehydration and environmental factors. As a result, the composition provides surprising performance benefits in reducing or removing fine lines and wrinkles, firming the skin, hydrating the skin and promoting a younger appearance.

The aloe leaf extract used in the compositions of the present invention may be obtained from commercial sources or harvested according to known collection and extraction procedures. The aloe leaf extract may be an extract of the leaves of aloe barbadensis. This extract provides a combination of vitamins, minerals and amino acids that has been found to hydrate and soothe skin irritations. One source for such an extract is Aloe Barbadensis Leaf Extract 200x, sold by Concentrated Aloe Corporation (Ormond Beach, Fl.)

According to one embodiment or composition of the invention, the aloe leaf extract is present in an amount ranging from 0.01% to 0.50% by weight of the topical skin care composition, and preferably from about 0.09% to 0.11% by weight of the topical skin care composition, and most preferably in an amount ranging from 0.05% to 0.10% by weight of the topical skin care composition.

The stable, oil based source of Vitamin C (ascorbic acid) used in the compositions of the present invention may be obtained from commercial sources. The stable, oil based source of Vitamin C has been found to provide anti-oxidant protection along with promotion of collagen synthesis to normalize uneven skin tone an diminish age spots. One source for such a stable, oil based source of Vitamin C (ascorbic acid) is BV-OSC, sold by Barnett Products Corporation (Edgewood Cliffs, N.J.)

According to one embodiment or composition of the invention, the stable, oil based source of Vitamin C (ascorbic acid) is present in an amount ranging from 0.01% to 10.0% by weight of the topical skin care composition, and preferably from about 0.095% to 3.0% by weight of the topical skin care composition, and most preferably in an amount ranging from 0.10% to 0.11% by weight of the topical skin care composition.

The palm tocotrienol/tocopherol complex used in the compositions of the present invention may be obtained from commercial sources. The palm tocotrienol/tocopherol complex is comprised predominately of four tocotrienol isomers (alpha, beta, gamma and delta) together with tocopherols. The palm tocotrienol/tocopherol complex has been found to maintain healthy skin especially by preventing oxidative stress and providing protection against UV-induced skin damage. One source for such a palm tocotrienol/tocopherol complex is Tocomin 50% C (L), sold by Carotech Inc.

According to one embodiment or composition of the invention, the palm tocotrienol/tocopherol complex is present in an amount ranging from 0.05% to 3.0% by weight of the topical skin care composition, and preferably from about 0.05% to 2.0% by weight of the topical skin care composition, and most preferably in an amount ranging from 0.09% to 0.11% by weight of the topical skin care composition.

The bioengineered biofunctional peptide used to boost the natural endogenous synthesis of CoQ10 used in the compositions of the present invention may be obtained from commercial sources. The bioengineered biofunctional peptide has been found to boost the natural endogenous synthesis of CoQ10 which helps to diminish the appearance of wrinkles and fine lines, and to retard the appearance of aging. One source for such a bioengineered biofunctional peptide is Peptide Q10 Biofunctional, sold by Ashland, Inc.

According to one embodiment or composition of the invention, the bioengineered biofunctional peptide is present in an amount ranging from 0.05% to 1.0% by weight of the topical skin care composition, and preferably from about 0.50% to 1.0% by weight of the topical skin care composition, and most preferably in an amount ranging from 0.45% to 0.55% by weight of the topical skin care composition.

The glutathione-biomimetic antioxidant peptide used in the compositions of the present invention may be obtained from commercial sources. The glutathione-biomimetic antioxidant peptide has been found to protect against glycation damage to the skin, to protect the skin from environmental stresses, and to prevent loss of skin resiliency. One source for such a glutathione-biomimetic antioxidant peptide is Quintescine IS, sold by Ashland, Inc.

According to one embodiment or composition of the invention, the glutathione-biomimetic antioxidant peptide is present in an amount ranging from 0.20% to 2.5% by weight of the topical skin care composition, and preferably from about 0.2% to 0.5% by weight of the topical skin care composition, and most preferably in an amount ranging from 0.225% to 0.275% by weight of the topical skin care composition.

The peptide inhibitor of elastase and MMP-1 used in the compositions of the present invention may be obtained from commercial sources. The peptide inhibitor of elastase and MMP-1 has been found to defend the skin collagen and elastin network against deleterious damage caused by aging and environmental factors such as UV radiation, pollution, and cigarette smoke. The peptide inhibitor of elastase and MMP-1 has also been found to reduce the symptoms of actinic aging and helps to increase the elasticity and firmness of the skin. One source for such a peptide inhibitor of elastase and MMP-1 is ECM-Protect BFG 100, sold by Lucas Meyer Cosmetics.

According to one embodiment or composition of the invention, the peptide inhibitor of elastase and MMP-1 is present in an amount ranging from 0.5% to 10.0% by weight of the topical skin care composition, and preferably from about 4.0% to 6.0% by weight of the topical skin care composition, and most preferably in an amount ranging from 4.5% to 5.5% by weight of the topical skin care composition.

The exopolysaccharide used in the compositions of the present invention may be obtained from commercial sources. The exopolysaccharide has been found to chelate heavy metals and to protect skin cells from UV and pollutants, thus decreasing premature aging and skin damage, and reducing skin sensitivity caused by urban pollution. One source for such an exopolysaccharide is EXO-P, sold by Lucas Meyer Cosmetics.

According to one embodiment or composition of the invention, the exopolysaccharide is present in an amount ranging from 0.3% to 5.0% by weight of the topical skin care composition, and preferably from about 0.5% to 3.0% by weight of the topical skin care composition, and most preferably in an amount ranging from 2.7% to 3.3% by weight of the topical skin care composition.

The knotgrass extract used in the compositions of the present invention may be obtained from commercial sources. The knotgrass extract is rich in flavonoids which have been found to protect dermis fibers from damage caused by UV and Infrared radiation from the sun. It has also been found to increase skin firmness and reduce the appearance of wrinkles and to prevent premature photo-aging of the skin. One source for knotgrass extract is ILIX-IR, sold by Lucas Meyer Cosmetics.

According to one embodiment or composition of the invention, the knotgrass extract is present in an amount ranging from 0.5% to 2.2% by weight of the topical skin care composition, and preferably from about 0.5% to 2.0% by weight of the topical skin care composition, and most preferably in an amount ranging from 1.8% to 2.0% by weight of the topical skin care composition.

The phyto-complex used in the compositions of the present invention may be obtained from commercial sources. The phyto-complex has been found to protect and improve skin radiance through the transformation of UV rays into a source of visible light. This helps reduce the appearance of fine lines and wrinkles, making skin to appear more radiant and youthful. One source for such a phyto-complex is Luminescine, sold by TRI-K Industries, Inc.

According to one embodiment or composition of the invention, the phyto-complex is present in an amount ranging from 1.0% to 5.0% by weight of the topical skin care composition, and preferably from about 3.0% to 5.0% by weight of the topical skin care composition, and most preferably in an amount ranging from 0.9% to 1.1% by weight of the topical skin care composition.

The oat avenanthramide extract used in the compositions of the present invention may be obtained from commercial sources. Oat avenanthramide extract has been found to be a natural anti-oxidant and anti-irritant that protects from UV exposure and reduces redness, inflammation and itching of the skin. One source for such an oat avenanthramide extract is CP Oat Avenanthramide Extract 902-3043, sold by Ceapro, Inc.

According to one embodiment or composition of the invention, the oat avenanthramide extract is present in an amount ranging from 0.1% to 3.0% by weight of the topical skin care composition, and preferably from about 0.3% to 2.0% by weight of the topical skin care composition, and most preferably in an amount ranging from 0.45% to 0.55% by weight of the topical skin care composition.

The oat β-glucan used in the compositions of the present invention may be obtained from commercial sources. The oat β-glucan has been found to penetrate and moisturize the skin, and to promote procollagen formation. One source for such a oat β-glucan is Colloidal Oatmeal Irradiated, sold by Oat Cosmetics or Charkit Chemical Corporation.

According to one embodiment or composition of the invention, the oat β-glucan is present in an amount ranging from 0.05% to 2.0% by weight of the topical skin care composition, and preferably from about 0.1% to 2.0% by weight of the topical skin care composition, and most preferably in an amount ranging from 0.225% to 0.275% by weight of the topical skin care composition.

The soy based complex used in the compositions of the present invention may be obtained from commercial sources. The soy based complex has been found to improve hydration, clarity, and firmness of the skin. The soy based complex has also been found to provide anti-flakiness and anti-wrinkle benefits. One source for such a soy based complex is Allosteris, sold by Barnet Products Corporation.

According to one embodiment or composition of the invention, the soy based complex is present in an amount ranging from 0.1% to 3.0% by weight of the topical skin care composition, and preferably from about 1.0% to 2.0% by weight of the topical skin care composition, and most preferably in an amount ranging from 0.9% to 1.1% by weight of the topical skin care composition.

The extract from dried apples used in the compositions of the present invention may be obtained from commercial sources. The extract from dried apples is a moisturizing saccharide complex that has been found to moisturize and smooth skin texture. One source for such an extract from dried apples is Botanimoist AMS, sold by Botanigenics, Inc.

According to one embodiment or composition of the invention, the extract from dried apples is present in an amount ranging from 0.5% to 10.0% by weight of the topical skin care composition, and preferably from about 0.5% to 5.0% by weight of the topical skin care composition, and most preferably in an amount ranging from 0.9% to 1.1% by weight of the topical skin care composition.

Other components may also be included in the compositions of various embodiments of the present invention. For example, purified water may be used as a diluent. Carpobol Ultrez 30 20, available from Lubrazol, which is a mixture of acrylates/C10-30 alkyl acrylate crosspolymer, may be used as a thickener and carrier. Ammonium acryloydimethyltaurateNP copolymer, sold by Clariant as Aristoflex AVC may be used as an emulsifier. Trisodium ethylenediamine disuccinate, sold by Innospec as Natriquest E30, may be used as a chelating agent. Propanediol dicaprylate/caprate, sold by Phoenix Chemical as Pelemol P-810, may be used as an emollient, and proanediol, sold by DuPont as Zemea, may be used as a humectant. A preservative, such as Linatural MBS-1, sold by Lincoln Fine Ingredients, may also be used. A pH modifier, such as aminomethyl propanol, sold as AMP Ultra PC 2000 by Angus, may also be used.

The invention is now more fully illustrated using the following example, which is not to be understood as limiting the invention to the embodiments described.

### Example 1

A topical cream for day use was prepared using the ingredients set forth in Table 1 below.

TABLE 1

| | Ingredient | % w/w |
|---|---|---|
| 1 | Water (USP) | 76.200 |
| 2 | Carbomer | 0.250 |
| 3 | Aloe Barbadensis Leaf Extract | 0.100 |
| 4 | Avena Sative (Oat) Kernel Flour | 0.250 |
| 5. | Ammonium Acryloyldimethyltaurate/VP Copolymer | 0.600 |
| 6 | Trisodium Ethylenediamine Disuccinate | 0.100 |
| 7 | Propanediol Dicaprylate/Caprate | 3.000 |
| 8 | Propanediol | 3.000 |
| 9 | Tocotrienols | 0.100 |
| | Tocopherol | |
| | Elaeis guineensis (Palm) Oil | |
| 10 | Tetrahexydecyl Ascorbate | 0.100 |
| 11 | Propanediol | 2.000 |
| | Ethylhexyglycerin | |
| | Potassium Sorbate | |
| 12 | Glycerin | 1.000 |
| | Hydrolyzed Verbascum Thapsus Flower | |
| | Citric Acid | |
| | Sodium Benzoate | |
| | Potassium Sorbate | |
| 13 | Pyrus Malus (Apple) Fruit Extract | 1.000 |
| | Glycerin | |
| 14 | Pentapeptide-34 Trifluoroacetate | 0.500 |
| | Sodium Benzoate | |
| 15 | Dipeptide-4 | 0.250 |
| | Glycerin | |
| 16 | Water | 5.000 |
| | Butylene Glycol | |
| | Dextran | |
| | Trifluoroacetyl Tripeptide-2 | |
| 17 | Water | 3.000 |
| | Butylene Glycol | |
| | Alteromonas Ferment Filtrate | |
| 18 | Water | 2.000 |
| | Glycerin | |
| | Polygonum Aviculare Extract | |
| | Potassium Sorbate | |
| | Sodium Benzoate | |
| 19 | Water | 1.000 |
| | Glycerin | |
| | Glycine (Soybean) Soja Extract | |
| | Phenoxethanol | |
| 20 | Avena Sativa (Oat) Extract | 0.500 |
| | Water | |
| | Glycerin | |
| | Potassium Sorbate | |
| 21 | Aminomethyl Propanol | 0.050 |

The day cream was prepared by mixing ingredients 1 through 21 in a sanitized stainless steel processing tank using moderate agitation until a homogenous cream suitable for application to the skin of a user is obtained. Mixing was performed at 30-50 hertz with propeller and side sweep agitation. After 1 hour of mixing, a top and bottom sample was evaluated for uniform polymer dispersion.

The resulting topical day cream was an opaque, viscous emulsion, off-white in color. The pH at 25 degrees centigrade was between 4.95-5.25, the viscosity was between 2000-6500 cPs as measured by a Brookfield viscometer, and the specific gravity at 25 degrees centigrade was between 0.982-1.022.

### Example 2

A topical cream for day use was prepared using the ingredients set forth in Table 2 below.

TABLE 2

| | Ingredient | % w/w |
|---|---|---|
| 1 | Water (USP) | 70.540 |
| 2 | Carbomer | 0.600 |
| 3 | Aloe Barbadensis Leaf Extract | 0.185 |
| 4 | Avena Sative (Oat) Kernel Flour | 0.225 |
| 5. | Ammonium Acryloyldimethyltaurate/VP Copolymer | 0.550 |
| 6 | Trisodium Ethylenediamine Disuccinate | 0.100 |
| 7 | Propanediol Dicaprylate/Caprate | 5.000 |
| 8 | Propanediol | 6.000 |
| 9. | Tocotrienols | 0.100 |
| | Tocopherol | |
| | Elaeis guineensis (Palm) Oil | |
| 10 | Tetrahexydecyl Ascorbate | 0.100 |
| 11 | Propanediol | 2.000 |
| | Ethylhexyglycerin | |
| | Potassium Sorbate | |
| 12 | Glycerin | 1.000 |
| | Hydrolyzed Verbascum Thapsus Flower | |
| | Citric Acid | |
| | Sodium Benzoate | |
| | Potassium Sorbate | |
| 13 | Pyrus Malus (Apple) Fruit Extract | 1.000 |
| | Glycerin | |
| 14 | Pentapeptide-34 Trifluoroacetate | 0.500 |
| | Sodium Benzoate | |
| 15 | Dipeptide-4 | 0.250 |
| | Glycerin | |
| 16 | Water | 5.000 |
| | Butylene Glycol | |
| | Dextran | |
| | Trifluoroacetyl Tripeptide-2 | |
| 17 | Water | 3.000 |
| | Butylene Glycol | |
| | Alteromonas Ferment Filtrate | |
| 18 | Water | 2.000 |
| | Glycerin | |
| | Polygonum Aviculare Extract | |
| | Potassium Sorbate | |
| | Sodium Benzoate | |
| 19 | Water | 1.000 |
| | Glycerin | |
| | Glycine (Soybean) Soja Extract | |
| | Phenoxethanol | |
| 20 | Avena Sativa (Oat) Extract | 0.500 |
| | Water | |
| | Glycerin | |
| | Potassium Sorbate | |
| 21 | Aminomethyl Propanol | 0.350 |

The day cream was prepared by mixing ingredients 1 through 21 in a sanitized stainless steel processing tank using moderate agitation until a homogenous cream suitable for application to the skin of a user is obtained. Mixing was performed at 30-50 hertz with propeller and side sweep agitation. After 1 hour of mixing, a top and bottom sample was evaluated for uniform polymer dispersion.

The resulting topical day cream was an opaque, viscous emulsion, off-white in color. The pH at 25 degrees centigrade was between 4.95-5.25, the viscosity was between 2000-6500 cPs as measured by a Brookfield viscometer, and the specific gravity at 25 degrees centigrade was between 0.982-1.022.

The examples presented above provide a process for making and using the various embodiments of the invention to enable a person skilled in the art to make and use the same. It will be understood that the various examples may be used to protect a user's face and other skin areas from the harmful effects of various environmental factors. It will also be understood that the various embodiments of the invention may be used in concert with other topical liquids, creams, sprays and the like without departing from the intended scope of the invention.

In another embodiment, the invention includes a method of treating skin comprising applying the topical skin care composition such as is described above to the skin. Typically, the topical skin care composition is applied to the skin, often the face, which may, for example, but not limited to, have wrinkles, fine lines, uneven tone, loss of firmness, surface roughness, dark circles, under-eye puffiness, sun damage, redness, dryness, irritation, enlarged ports and combinations of all or some of the above. Alternatively, the topical skin care composition may be applied to the skin to prevent the occurrence of the various problems described above.

The novel compositions of the present invention are used by subjects desiring to obtain the benefits noted above, including the hydration of their skin, increasing the skins resiliency and radiance, and proving protection against inflammation-related signs of aging, such as fine lines and wrinkles. Use of the various compositions of the invention may decrease the size of skin pores, even out the tone of the skin, and improve the texture of the skin.

Typically, a person using the compositions of the invention apply them to the their skin in amounts that obtain one or more of the noted benefits. For example, the compositions of the invention may be applied to a skin area so as to improve the texture of the skin, reduce pore size, or hydrate dry skin. Alternatively, the compositions may be applied to prevent damage to the skin caused by environmental facts that sunscreen cannot protect against, such as, for example, damage caused by inflammation and infrared radiation.

The amount used is typically sufficient to obtain coverage of a desired area of the skin, such as the face, with a single application. The compositions of the various embodiments of the present invention may also be used over the course of a period of time, with the amount of the compositions used including the amount used during repeated applications. For example, the compositions of the various embodiments of the present invention may be applied to a desired area of the skin on a daily basis, either once a day or several times per day, over the course of days, weeks, months or any time period desired by the user. The various compositions of the embodiments of the invention are typically considered to be light-weight, easily absorbed and layer cleanly under makeup or other substances applied to the skin without pilling or feeling heavy.

The skin care compositions of the various embodiments of the present invention may be formulated to be used on an “as needed” basis, or they may be formulated for application at specific times of the day, or multiple times during the time the user is awake. They may also be formulated for use on an every other day, weekly, monthly or other basis. The compositions of the various embodiments of the present invention may also be formulated for application by the fingers of the user, or they may be formulated for application by some application means, such as, for example, a soft pad or other applicator well known in the art. The compositions of the various embodiments of the present invention may also be used as part of a skin treatment regimen, and may also be used in conjunction with other skin creams and makeup.

In one embodiment, a user of a skin care composition in accordance with the present invention cleanses his/her skin and gently pats the skin dry. A thin, even layer of the skin care composition in accordance with the present invention is applied to the face, neck, or other portions of the body. The skin care composition is then gently massaged into the skin. This process may be performed every morning, for example.

While particular embodiments of the present invention have been described, it is understood that various different modifications within the scope and spirit of the invention are possible. The invention is limited only by the scope of the appended claims.

We claim:

1. A topical skin care composition comprising:
(a) an oat derived avenanthramide;
(b) an aloe barbadensis leaf extract;
(c) a source of vitamin E;
(d) a source of biofunctional peptides;
(e) an ascorbic acid source;
(f) a blend of anti-inflammation and anti-oxidant components; and
(g) a cosmetically acceptable carrier.

2. The topical skin care composition of claim 1, wherein the source of vitamin E is a palm tocotrienol/tocopherol complex.

3. The topical skin care composition of claim 1, wherein the aloe barbadensis extract is present in the amount of 0.09% by weight to 0.11% by weight.

4. The topical skin care composition of claim 1, wherein the source of ascorbic acid is tetrahexydecyl ascorbate.

5. The topical skin care composition of claim 1, wherein the source of ascorbic acid is present in the amount of 0.05% by weight to 1.0% by weight.

6. The topical skin care composition of claim 1, wherein the biofunctional peptides are present in the amount of 0.05% by weight to 1.0% by weight.

7. The topical skin care composition of claim 1, wherein the oat derived avenanthramide extract is present in the amount of 0.1% by weight to 3.0% by weight.

8. The topical skin care composition of claim 1, wherein the oat derived avenanthramide extract is present in the amount of 0.45% by weight to 0.55% by weight.

9. The topical skin care composition of claim 1, further comprising a soy based active complex.

10. The topical skin care composition of claim 1, further comprising a phyto-complex component.

11. The topical skin care composition of claim 1, further comprising a source of flavonoids.

12. The topical skin care composition of claim 11, wherein the source of flavonoids includes knotgrass extract.

13. The topical skin care composition of claim 1, wherein the blend of anti-inflammation and anti-oxidant components includes colloidal oatmeal.

14. The topical skin care composition of claim 1, wherein the blend of anti-inflammation and anti-oxidant components includes tocopherol.

15. The topical skin care composition of claim 1, wherein the blend of anti-inflammation and anti-oxidant components includes tocopherol and tocotrienol.

16. The topical skin care composition of claim 1, wherein the source of biofunctional peptides includes pentapeptide-34 trifluoroacetate.

17. The topical skin care composition of claim 1, wherein the source of biofunctional peptides includes dipeptide-4.

18. The topical skin care composition of claim 1, further including an exopolysaccharide.

19. The topical skin care composition of claim 1, further including an apple extract.

20. A method of treating the skin, comprising applying to the outer surface of the skin the composition according to claim 1.
